(19) United States
(12) Patent Application Publication
(10) Pub. No.: US 2008/0119842 A1
(43) Pub. Date: May 22, 2008

(54) Electro-adhesive tissue manipulation method

(75) Inventors: Daniel Palanker, Sunnyvale, CA (US); Alexander Vankov, Menlo Park, CA (US)

Correspondence Address: MORRISON & FOERSTER LLP, 755 PAGE MILL RD, PALO ALTO, CA, 94304-1018, US

(73) Assignee: The Board of Trustees of the Leland Stanford Junior University, Palo Alto, CA (US)

(21) Appl. No.: 11/974,611

(22) Filed: Oct. 12, 2007

Related U.S. Application Data
(62) Division of application No. 10/871,697, filed on Jun. 18, 2004, now abandoned.
(60) Provisional application No. 60/479,825, filed on Jun. 18, 2003.

(51) Int. Cl. A61B 17/28

(52) U.S. Cl. 606/34; 606/205

(57) ABSTRACT

An electro-adhesive tissue manipulation method capable of manipulating tissue with a single conducting element is provided. The manipulator includes a conducting element, a pulse generator and a controller capable of generating a first and a second pulse on demand. The first pulse generates an adhesive state between the conducting element and the tissue layer strong enough to manipulate the tissue layer. The second pulse, which has higher pulse energy than the first pulse, generates a non-adhesive state to detach the adhered tissue layer from the conducting element. The electro-adhesive device could be combined with a medical instrument to enhance the capabilities of the medical instrument so that it can manipulate tissue. Thereby tissue can be manipulated with a single tip of a conducting element, without folding and piercing of the tissue, thus avoiding damage to the tissue.

### CROSS-REFERENCE TO RELATED APPLICATIONS

This application is a divisional application of U.S. patent application Ser. No. 10/871,697, filed on Jun. 18, 2004, which in turn is based on and claims priority from U.S. Provisional Patent Application 60/479,825 filed on Jun. 18, 2003, both incorporated herein by reference.

### STATEMENT REGARDING FEDERALLY SPONSORED RESEARCH OR DEVELOPMENT

This invention was supported in part by grant number NIH R01-EY012888 from the National Institutes of Health (NIH). The U.S. Government has certain rights in the invention.

### FIELD OF THE INVENTION

The present invention relates generally to medical devices. More particularly, the present invention relates to devices for tissue manipulation.

### BACKGROUND

Mechanical forceps or tweezers are widely used for manipulation of tissue in microsurgery in general and in opthalmology in particular. Capturing a thin and evasive membrane is a difficult task since such membranes easily escape the grip of the forceps due to even a minor flow of water introduced during closure of the forceps. Another difficulty is in grasping a thin membrane strongly attached to the underlying tissue. The most difficult part of such procedure is in initial separation of the membrane, which will then allow for a strong grip of the micro-tweezers holding it from two sides. Attempts of performing this procedure often lead to piercing and otherwise damaging the underlying tissue. Accordingly, there is a need for better tissue manipulation devices. It would for instance be desirable to have a micromanipulator that could attach to a tissue on a push of a button and release it on demand. It would also be desirable to have a tissue manipulator that makes it possible to access tissue only from one side.

### BRIEF SUMMARY OF THE INVENTION

The present invention is an electro-adhesive tissue manipulator. The electro-adhesive manipulator includes a conducting element and an electrical means capable of providing a first pulse and a second pulse to the conducting element. The first pulse generates an adhesive state between the conducting element and a tissue layer strong enough to manipulate the tissue layer with the electro-adhesive manipulator. The second pulse, which has a higher pulse energy than the first pulse, generates a non-adhesive state to the adhered tissue layer to detach the adhered tissue layer from the conducting element. In a preferred embodiment the duration of the first pulse varies between 10 microseconds to 10 milliseconds. The first and second pulse could be a single pulse or a burst of pulses. The pulse energy of the first pulse is below the threshold energy required for formation of a complete vapor cavity around the conducting element. The second pulse should have sufficient pulse energy to generate a vapor cavity around the conducting element that is in contact with the tissue layer to detach the adhered tissue layer from the conducting element. The electro-adhesive device of the present invention could be combined with a medical instrument to enhance the capabilities of the medical instrument so that it can manipulate tissue. The advantage of the present invention, in contrast to mechanical tools, is that tissue can be manipulated without folding and piercing thus avoiding damage to the underlying tissue. This feature makes most of the area of a membrane available for operation or intervention.

### BRIEF DESCRIPTION OF THE DRAWINGS

The objectives and advantages of the present invention will be understood by reading the following detailed description in conjunction with the drawings, in which:

FIG. 1 shows an example of an electro-adhesive tissue manipulator according to the present invention.

FIG. 2 shows an example of a membrane that is being elevated by an electro-adhesive tissue manipulator according to the present invention.

FIG. 3 shows an example of the pulses and their energy to attach and detach tissue to the conductive element according to the present invention.

FIG. 4 shows an example of a pulse and a burst of pulses according to the present invention.

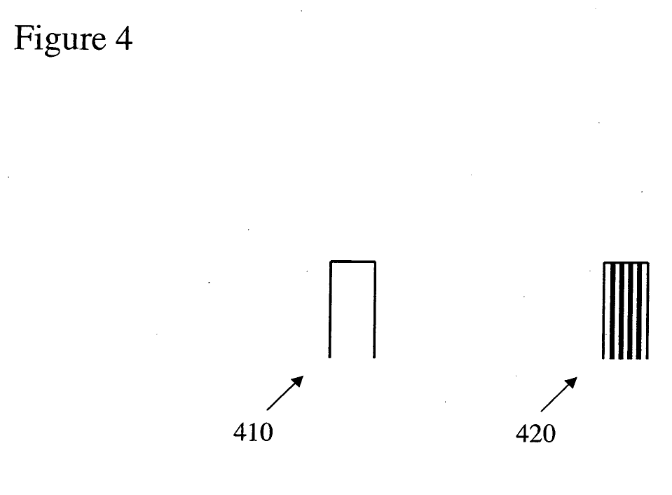

FIG. 5 shows an example of a damage zone of about two cellular layers in width is present in front of the conductive element after staining the tissue with propidium iodide according to the present invention.

FIG. 6 shows examples of the shape of the conductive element according to the present invention.

FIG. 7 shows an example of an electro-adhesive tissue manipulator combined with a needle according to the present invention.

FIG. 8 shows an example of an electro-adhesive tissue manipulator combined with a conventional forceps according to the present invention.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

The present invention is an electro-adhesive tissue manipulator that is able to attach to a tissue on demand and release it on demand. The electro-adhesive tissue manipulator could be used to manipulate any kind of biological tissue layer during, for instance, surgical procedures, tissue implants, interventions (including drug, agent or antibiotic interventions), or the like. As it will be clear by reading the description, the electro-adhesive tissue manipulator will make it possible to manipulate tissue by accessing the tissue from only one side. This is in contrast to the use of tweezers or forceps since these will require access of a tissue from two sides, i.e. pinch or grip the tissue.

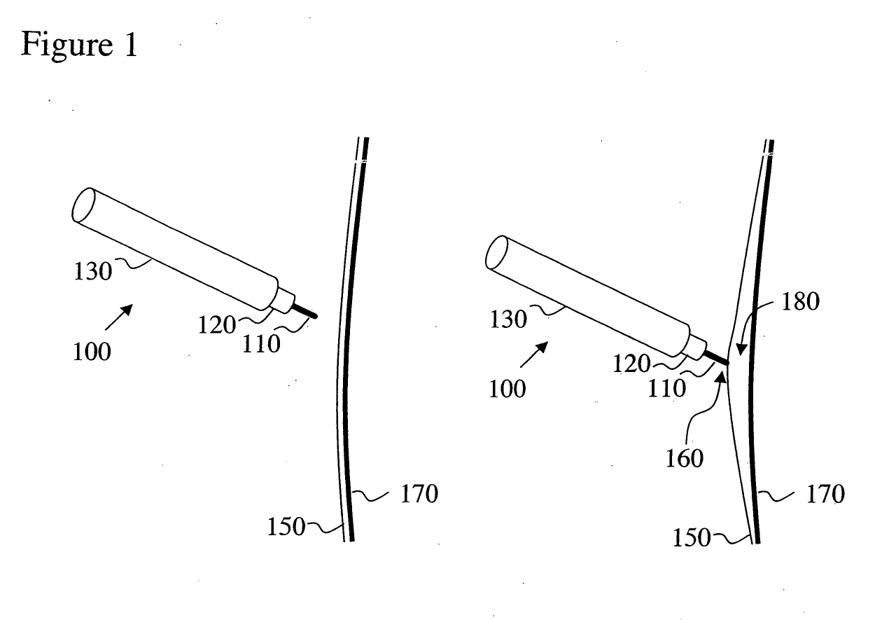

FIG. 1 shows an electro-adhesive tissue manipulator 100 according to the present invention. Electro-adhesive tissue manipulator 100 is composed of an insulated probe 120 with a protruding conductive element 110. Conductive element 110 serves as an active electrode and could be made out of a metal wire, a tungsten filament, or any type of material that has conductive properties. A second electrode is used as a return electrode. The return electrode is typically much larger than the active electrode and its location in the operation field is not critical. In the example of FIG. 1, the second electrode could be a needle, which hosts insulated probe 120 and conductive element 110. In one embodiment the following parameters were used: a 20 Gauge needle (about 0.92 mm), an insulator (e.g. glass or plastic; about 0.64 mm in diameter) and a wire of about 50 micrometers in diameter and 1 mm long. However, the invention is not limited to these dimensions. The conductive element could range from about 10 micrometers to about 10 millimeters in diameter.

Electro-adhesive tissue manipulator 100 is activated by an electrical means (e.g. a pulse generator) capable of providing a first (electrical) pulse and a second (electrical) pulse between conducting element 110 and the return electrode 130. Preferably the manipulator has a control means in communication with e.g. buttons on the manipulator, a foot-pedal connected to the manipulator or even a voice recognition means to control the generation of the pulses. Once conducting element is placed in contact with a tissue layer 150 and first pulse is generated on demand, the state of adhesiveness of tissue layer 150 is changed as a result. The adhesiveness of tissue is created by partial denaturation of proteins in the proximity to the conductive element. This effect is induced either by high electric field and/or heating. This change in adhesiveness creates an adhesive bonding 160 between conductive element 110 and tissue layer 150 through which electro-adhesive tissue manipulator 100 is capable of manipulating tissue layer 150. Tissue layer 150 could be elevated from an underlying tissue layer 170. In one example a cavity 180 between tissue layer 150 and underlying tissue layer 170 is created. Cavity 180 could be useful for implantation, intervention or delivery of an agent, a drug or an antibiotic. The adhesive bonding is remarkably strong and allows one to move a tissue layer in any direction as well as to elevate it away from underlying tissue layer(s). There are no pulses required after the adhesion is achieved; tissue can be kept to the conducting element as long as the second pulse is not applied.

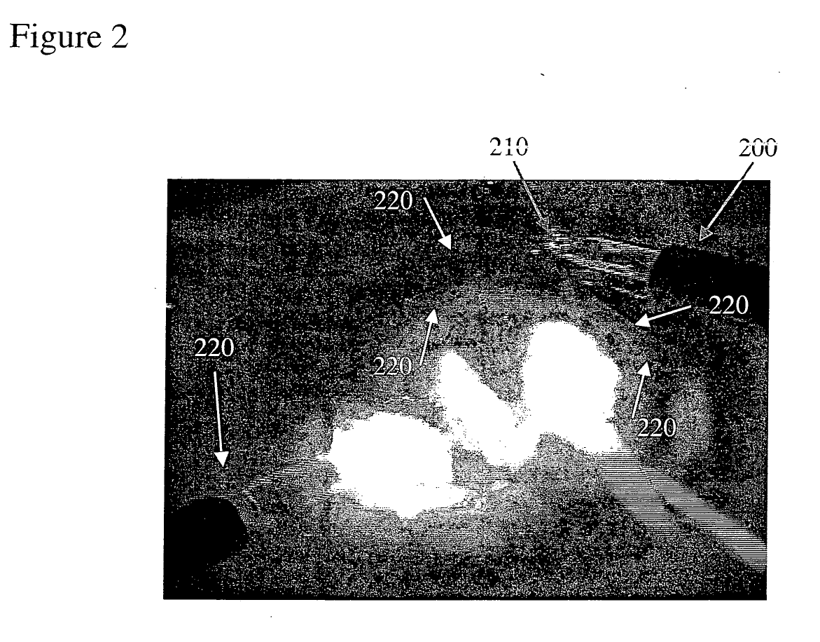

FIG. 2 shows a membrane 220 that is elevated by electro-adhesive tissue manipulator 200 when attached to conducting element 210. FIG. 2 shows an illumination probe 220 to highlight the elevated membrane.

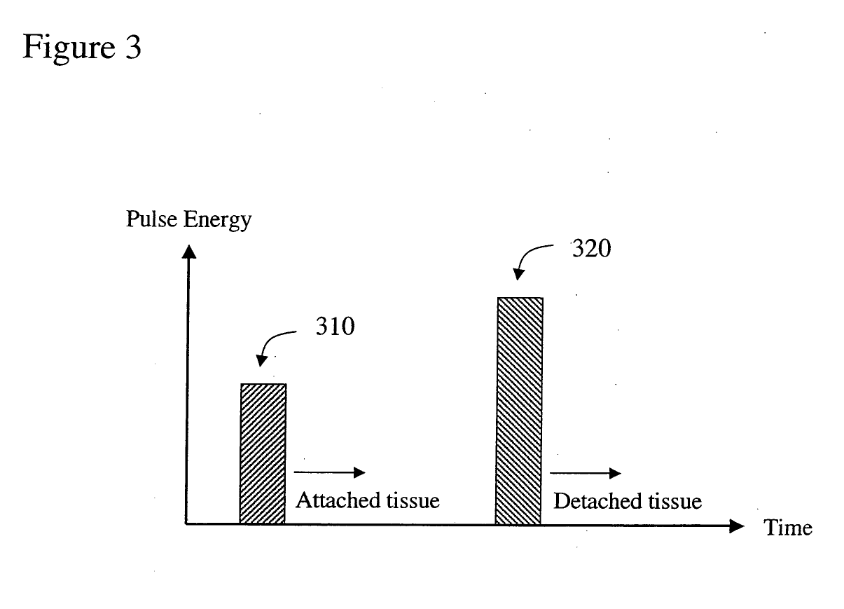

To establish electro-adhesion, pulse duration of the first pulse 310 (See FIG. 3) can vary between about 10 microseconds to about 10 milliseconds. More specifically the duration of the first pulse varies from about 1 microsecond to about 0.5 milliseconds. Pulse duration is limited on a long side by heat diffusion; i.e. to avoid thermal damage beyond 100 μm the pulse duration should preferably not exceed 10 ms. Pulse energy should be below the threshold energy required for formation of a complete vapor cavity around the conducting element. A complete vapor cavity will disconnect the conducting element from the tissue and prevent adhesion. In fact, the effect of vapor cavity is used to disconnect the attached tissue from the conducting element (see below).

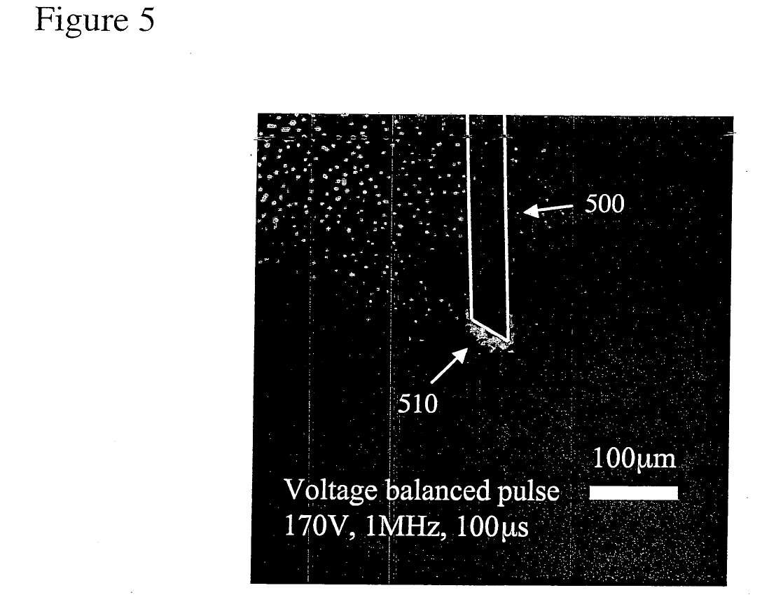

The first pulse could be a single pulse 410 or a burst of shorter pulses 420 with a frequency that could vary between about 0.1 kHz to 10 Mhz. The first pulse could be a unipolar or a charge-balanced or voltage-balanced bipolar burst of pulses. Application of such pulse or a few pulses when the probe is held in contact with a tissue layer induces adhesion of the tissue to the metal surface, and so the tissue can be lifted and manipulated. In one embodiment pulse parameters are 200V with a 100 microsecond pulse duration. Voltage should be above 50 V, but below 500 V, since threshold of plasma formation is somewhere between 200 to 400 V, depending on pulse parameters and electrode configuration. To minimize the tissue damage induced by electroporation a voltage-balanced train of pulses could be applied. At optimal settings the damage does not exceed one or two layers of cells 510 adjacent to the probe 500, as shown in FIG. 5.

To detach the tissue layer from the conducting element, a stronger (in terms of energy) second pulse 320 needs to be applied, such that it creates a complete vapor cavity around the probe thus detaching the tissue from the conducting element. The second pulse could also be a single pulse 410 or a burst of shorter pulses 420 with a frequency that could vary between about 0.1 kHz to 10 Mhz. The duration of the second pulse could be between about 10 microseconds to about 10 milliseconds. More specifically the duration of the second pulse varies from about 1 microsecond to about 0.5 milliseconds. The second pulse could also be a unipolar or a charge-balanced or voltage-balanced bipolar burst of pulses. To minimize the tissue damage induced by electroporation a voltage-balanced train of pulses can be applied.

To establish successful adhesion of a conducting element to a tissue layer, it is important to maintain the surface of the conducting element clean of biological debris. If the conducting element does get contaminated, i.e. coated with a layer of coagulated proteins and other materials the conducting element can easily be cleaned without withdrawal from the surgical field. This can for instance be accomplished by application of few pulses in the plasma-mediated cutting regime. These pulses remove all the debris from the conducting element. To avoid tissue damage during this procedure the conducting element should be withdrawn from tissue by a certain distance. In one embodiment the conducting element was withdrawn at least 0.1 mm; distance larger than the width of the typical damage zone in cutting regime.

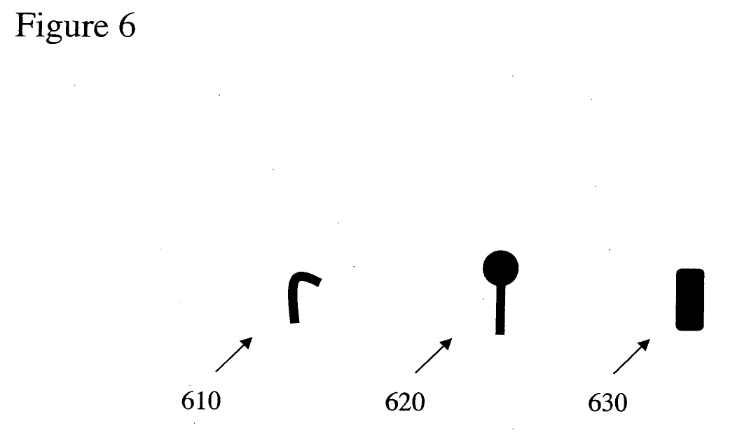

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For instance, the conducting element could take any type of shape, but is preferably dull. FIG. 6 shows some examples of different shapes of conductive elements such as a hooked shape 610, a ball-shape 620, or a rectangular shape 630, which should all be regarded as illustrative rather than limiting to the scope of the invention.

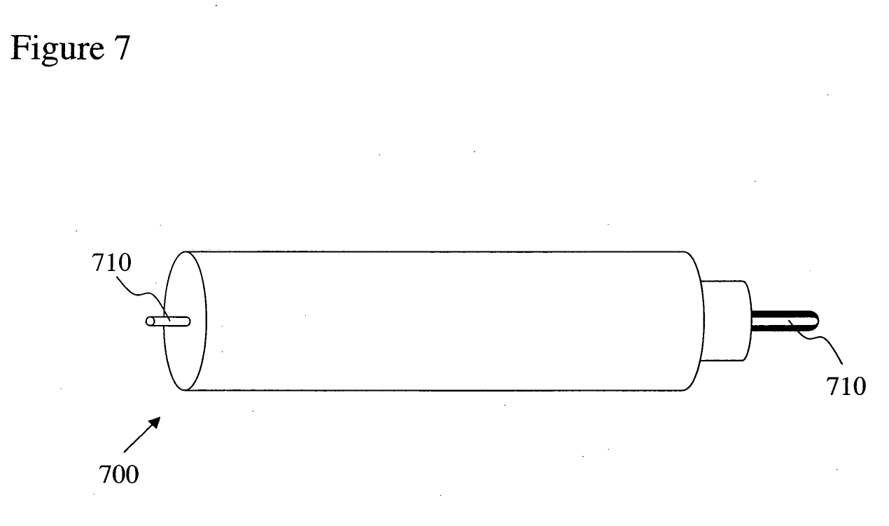

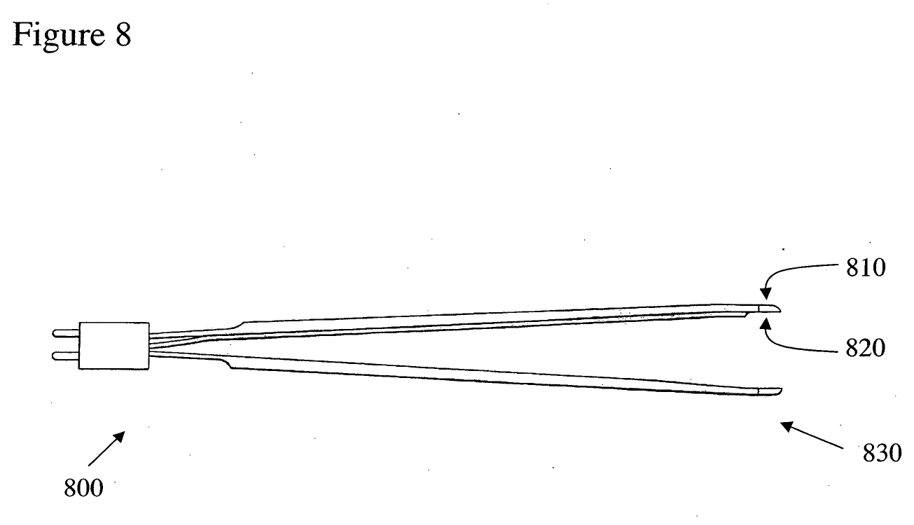

Conventional medical instruments could be combined with electro-adhesive tissue manipulation features as embodied in the present invention by coating them with isolating material and exposing a part that will be used as an active electrode. FIG. 7 shows electro-adhesive tissue manipulator 700 combined with a needle 710 for injection of a liquid, agent, drug of antibiotic under an elevated tissue layer to enhance tissue separation. All the surface of the needle may be exposed and used as an active conductive element (electrode), or alternatively, a part of its surface might be coated and part be exposed. FIG. 8 shows a conventional forceps 800 that can be coated with insulating material and a strip of the arm (e.g. at location 810 or 820) can be exposed to use it as a conducting element (electrode) to develop an electrical forceps embodying the features of the present invention. To increase the mechanical force, a second (conventional) arm of the forceps may be used for mechanical grasp of the tissue as soon as it is detached from the underlying tissue. The second arm 830 of forceps 800 can also be made as an active conducting element (electrode). This combination can be used, for example, for cutting of tissue attached to the first arm. Since tissue is approached from only one side a device embodying the features of the present invention does not have to have a sharp-pointed end, as conventional micro-forceps typically do. Lack of the sharp apex makes it safer with respect to occasional or unintended piercing of tissue.

In addition to the types of applications discussed herein the electro-adhesive tissue manipulator could further be used for peeling or lifting thin membranes, for example in vitreoretinal surgery. Another application of the electro-adhesive tissue manipulator could be attaching a lens holder to a surface of an eye for posterior pole surgery (replacing a current suturing procedure). For this application, the lens holder should have an active electrode or an array of active electrodes on its periphery, which will induce adhesion to sclera outside cornea (in order to avoid potential damage to corneal surface). Yet another application could include attaching an implant to tissue for anchoring or attaching temporary patches to tissue surface during operation. Still another application could include attaching tissue to the scaffold or reconnecting two ends of a cut blood vessel using a conductive stent.

All such variations are considered to be within the scope and spirit of the present invention as defined by the following claims and their legal equivalents.

1-6. (canceled)

7. A method of manipulating a tissue layer, comprising the steps of:
(a) providing a conducting element;
(b) applying a first pulse to said conducting element wherein said first pulse adheres said conducting element to said tissue layer; and
(c) manipulating said tissue layer.

8. The method as set forth in claim 7, further comprising the step of applying a second pulse to said conducting element to detach said adhered tissue layer from said conducting element.

9. The method as set forth in claim 7, wherein the duration of said first pulse or said second pulse ranges from 10 microseconds to 10 milliseconds.

10. The method as set forth in claim 7, wherein the duration of said first pulse or said second pulse ranges from about 1 microsecond to about 0.5 milliseconds.

11. The method as set forth in claim 7, wherein said first pulse or said second pulse is a monophasic or biphasic pulse burst with a frequency between about 0.1 kHz to 10 Mhz.

12. The method as set forth in claim 7, wherein the pulse energy of said first pulse is below the threshold energy required for formation of a complete vapor cavity around said conducting element.

13. The method as set forth in claim 7, wherein said second pulse generates a vapor cavity around said conducting element that is in contact with said tissue layer to detach said tissue layer from said conducting element.

14-17. (canceled)
